(19) United States
(12) Patent Application Publication
(10) Pub. No.: US 2005/0053666 A1
(43) Pub. Date: Mar. 10, 2005

(54) Antibody-containing particles and compositions

(76) Inventors: Stelios Tzannis, Newark, CA (US); Robert A. Platz, Half Moon Bay, CA (US); Bhas A. Dani, San Mateo, CA (US)

Correspondence Address: NEKTAR THERAPEUTICS, 150 INDUSTRIAL ROAD, SAN CARLOS, CA, 94070, US

(21) Appl. No.: 10/714,575

(22) Filed: Nov. 14, 2003

Related U.S. Application Data
(60) Provisional application No. 60/437,249, filed on Dec. 31, 2002.

(51) Int. Cl. A61K 39/395; A61K 9/14

(52) U.S. Cl. 424/489; 424/178.1; 424/141.1

(57) ABSTRACT

A composition is provided comprising antibody-containing particles. These particles can be used to form antibody-containing powders useful for reconstitution with a suitable diluent. The reconstituted compositions, in turn, comprise an antibody in an amount suited for delivery by injection, such as subcutaneous injection. Methods for preparing the various compositions as well as methods of use are also provided.

### CROSS REFERENCE TO RELATED APPLICATION

This application claims the benefit of priority to provisional application Ser. No. 60/437,249, filed Dec. 31, 2002, which is incorporated herein by reference.

### FIELD OF THE INVENTION

The present invention relates generally to antibody-containing particles that can form powdered compositions. These compositions, in turn, can be reconstituted with a diluent, thereby forming a reconstituted composition that is suited for, among other things, subcutaneous administration. In addition, the invention relates to methods for preparing reconstituted compositions as well as to methods for administering the reconstituted compositions to patients.

### BACKGROUND OF THE INVENTION

Antibodies are relatively large macromolecules that are produced by living organisms such as mammals. Antibodies are often, although not necessarily, secreted as part of an immune response to the presence of a foreign protein within the organism. The antibodies so formed have the ability to specifically bind to the foreign protein, thereby forming an antibody-foreign protein complex that can be cleared or otherwise neutralized by the organism. Thus, antibodies play an important role in the immune response of many organisms.

Scientists and researchers have found other uses of antibodies. For example, clinicians rely on the specificity of antibodies to bind to certain proteins in order to provide protein-detection tests. In the most basic antibody-based protein-detection test, a sample that may contain the protein of interest is immobilized onto a substrate. Thereafter, a solution containing labeled-antibodies (e.g., radiolabeled antibodies) is placed in contact with the protein-bound substrate, thereby allowing the labeled antibody to bind to the protein on the substrate. A washing step is then performed in order to remove any unbound labeled antibodies. Detection of the labeled antibody by, for example, exposure to an appropriate film, reveals that the protein of interest was present in the sample. Variations of this approach using radiolabels and other detectable labels are used in a number of detection and diagnostic assays such as home pregnancy tests and Western blots.

Scientists also rely on the specificity of antibodies to recover a protein of interest from a mixture containing several different proteins. In this approach, antibodies having affinity to the same protein of interest are immobilized on a substrate in the form of a column (commonly referred to as an “affinity column”). A mixture of proteins is passed through the column with the result that any protein of interest is retained in the column through antibody-protein binding. The protein in relatively pure form can then be retrieved by contacting the column with a suitable agent (e.g., acid) to release the bound protein, thereby allowing recovery of a relatively high concentration of the protein.

More recently, scientists have relied on the specificity of antibodies in the treatment of patients suffering from certain conditions or diseases. Although initially showing great promise in the treatment of patients, antibodies for therapeutic use have encountered a number of problems that have limited the widespread adoption of this therapeutic approach. In particular, difficulties have been encountered in providing antibodies intended for therapeutic use in a form suitable for administration to a patient.

For example, antibodies are unsuited for absorption through the gastrointestinal tract because the proteinaceous character of antibodies exposes these agents to unacceptably high degradation. Thus, other modes of administration are required.

Injection of therapeutic antibodies bypasses the problems associated with degradation of antibodies in the gastrointestinal tract. Injection of antibodies, however, is fraught with significant challenges. In particular, the typically low potency of antibodies often requires that they be administered in relatively large amounts per dose in order to effect pharmacologically effective levels in vivo. Inasmuch as subcutaneous injections are concerned, large doses of active agents such as antibodies are not easily delivered subcutaneously given the limitations of the acceptable volumes for subcutaneous administration (typically 0.5-1 mL) associated with this route of administration. Thus, with respect to subcutaneous injection of antibodies, a relatively large amount (i.e., dose) of the antibodies must be present in a relatively small volume of formulation. Moreover, it is generally preferred to inject smaller volumes (subcutaneously or otherwise) in order to avoid problems associated with fluid balance, blood pressure, osmotic imbalance and so forth.

Thus, subcutaneous injections of antibodies typically require concentrations well in excess of 100 mg/mL, and often in the 200-500 mg/mL range. These relatively high concentration requirements represent a significant challenge for all macromolecules, and particularly for antibodies. In particular, aggregation of antibody molecules—due to, in part, the inherently low aqueous solubility of antibodies—is particularly problematic, especially when relatively high concentrations of antibodies are required. Moreover, aggregation of antibodies becomes more pronounced as their concentration increases. See U.S. Pat. No. 6,267,958.

The difficulties associated with aggregation present themselves not only during formulation, but also during manufacturing as well. Furthermore, aggregation often occurs upon storage (particularly at room temperature). Solid dosage forms could, in principle, overcome the shelf-life constraints of solution-based formulations. Such solid forms, however, would still be required to enable drug stability upon reconstitution at high concentration and to provide a reasonably short and efficient reconstitution. Finally, any dosage form would need to have low viscosity to allow easy and reproducible syringeability from, for example, 26-29G needles.

U.S. Patent Application Publication US 2002/0136719 proposes using the crystalline form of whole antibodies and antibody fragments in order to provide stabilized formulations of these proteins. The problem with this approach, however, is that providing a crystalline antibody and/or antibody fragment introduces additional steps and/or complexity associated with the manufacture of the formulation. Moreover, crystalline antibodies that are injected in suspension form can be prone to difficulties of storage, dose adjustment, and administration typically encountered with crystals.

Although lyophilization-based formulations of antibodies have been proposed in, for example, U.S. Pat. No. 6,267,958, such formulations are known to have relatively long reconstitution times, which present coordination issues with respect to preparing both the formulation as well as the patient for administration of the reconstituted dosage form. Consequently, additional formulation approaches are needed. The present invention is therefore directed to provide, among other things, antibody-containing formulations that have relatively quick reconstitution times so as to solve problems associated with administering antibodies.

### SUMMARY OF THE INVENTION

Accordingly, it is a primary object of the invention to provide a composition comprising antibody-containing particles, wherein the particles have a median mass diameter of greater than 7.5 μm and less than about 100 μm.

It is a further object of the invention to provide a reconstituted composition comprising an antibody in an amount of from about 25 mg/mL to about 1000 mg/mL, a diluent and an optional excipient, wherein the reconstituted composition is formed from a powder comprised of antibody-containing particles and the optional excipient.

It is still yet another object of the invention to provide such compositions wherein the particles are prepared by spray drying.

It is another object of the invention to provide such compositions wherein the antibody is an IgG type antibody.

It is a further objection of the invention to provide such compositions wherein the optional excipient is present.

It is an additional object of the invention to provide a method for preparing a reconstituted composition comprising the steps of providing a powder comprised of antibody-containing particles and adding a diluent in order to form the reconstituted composition, wherein the antibody is present in the reconstituted composition in an amount of from about 25 mg/mL to about 1000 mg/mL.

It is a further object of the invention to provide such a method wherein the reconstituted composition comprises an excipient.

It is still another object of the invention to provide such a method wherein the excipient is present in the powder.

It is yet a further object of the invention to provide such a method wherein the excipient is added with or after the step of adding the diluent.

It is still a further object of the invention to provide such a method wherein the reconstituted composition becomes visually clear within about 15 minutes of adding the diluent.

It is still a further objection of the invention to provide such a method wherein the reconstituted composition becomes visually clear within about 10 minutes of adding the diluent.

It is another object of the invention to provide such a method wherein the reconstituted composition becomes visually clear within about 5 minutes of adding the diluent.

It is yet another object of the invention to provide such a method wherein the antibody is present in the reconstituted composition in an amount of from about 25 mg/mL to about 250 mg/mL.

It is a further object of the invention to provide a method for treating a patient comprising administering, via injection, a reconstituted composition described herein.

Additional objects, advantages and novel features of the invention will be set forth in the description that follows, and in part, will become apparent to those skilled in the art upon the following, or may be learned by practice of the invention.

In one embodiment then, a composition is provided comprising antibody-containing particles. The antibody-containing particles preferably have a mass median diameter (MMD) of greater than 7.5 μm and less than 100 μm. As will be further explained in detail below, the antibody-containing particles are typically, although not necessarily, prepared by spray-drying a liquid comprising the antibody. Particles formed in this way are conventionally referred to as “spray-dried particles.” A collection of spray-dried particles, in turn, is conventionally referred to as a “spray-dried powder,” in contrast to other powders formed from alternative methods.

Preferably, the antibody used in accordance with the invention is noncrystalline. Advantageously, the present invention is fully compatible with noncrystalline antibodies, thereby avoiding the extra steps and expense of providing antibodies in crystalline form. In some circumstances, the antibodies can be present in amorphous form, substantially amorphous form, or partially amorphous form.

In another embodiment, a reconstituted composition is provided comprising an antibody in an amount of from about 25 mg/mL to about 1000 mg/mL, a diluent and an optional excipient, wherein the reconstituted composition is formed from a powder (typically a spray-dried powder) comprised of the antibody and the optional excipient. Preferably, the reconstituted composition is prepared such that it is in sterile form.

The antibody can comprise any antibody and the invention is not limited in this regard. Advantageously, the reconstituted composition comprises a relatively high concentration of the antibody. Moreover, the particles, powders and reconstituted compositions possess a minimal amount of aggregates of the antibodies.

Another embodiment of the invention provides a method for preparing a reconstituted composition comprising the steps of providing a powder (again, typically, although not necessarily a spray-dried powder) comprised of an antibody and adding a diluent in order to form the reconstituted composition, wherein the antibody is present in the reconstituted composition in an amount of from about 25 mg/mL to about 1000 mg/mL. Optionally, an excipient may be present in the reconstituted composition. When present, the excipient can be (a) located in each antibody-containing particle and/or (b) located in the spray-dried powder, but distinct and separate from the antibody-containing particles and/or (c) added with or after the step of adding the diluent. Combinations of any of the foregoing are also envisioned.

When a spray-dried powder is desired, the step of providing the powder can be achieved by spray-drying a liquid feed mixture comprising the antibody, as described in more detail below. One of the benefits of the invention is that the reconstitution time (e.g., the time from adding the diluent to achieving visual clarity within the reconstituted composition) is relatively short, thereby eliminating the complexities associated with coordinating composition preparation and patient administration.

In another embodiment of the invention, a method for administering the reconstituted compositions to a patient is provided. This method comprises administering to the patient a therapeutically effective amount of the antibody, preferably present in a reconstituted composition as described herein. In this embodiment, a patient suffering from a condition that is responsive to administration of the antibody is administered a therapeutically effective amount of antibody via injection, e.g., subcutaneous injection.

### BRIEF DESCRIPTION OF THE DRAWINGS

FIG. 1A shows the percent monomer analysis (using size-exclusion chromatography-high performance liquid chromatography, “SEC-HPLC”) of an IgG-containing formulation before spray drying (“Before SD”) and after spray drying (“After SD”), as described in the Examples.

FIG. 1B is a chromatogram for a lyophilized human IgG starting material after reconstitution at 5 mg/mL, as further explained in the Examples.

FIG. 1C is a chromatogram for a spray-dried human IgG formulation after reconstitution at 5 mg/mL, as further explained in the Examples.

FIG. 2A shows percent monomer analysis (SEC-HPLC) of a lyophilized material and a spray-dried formulation at various concentrations, as further explained in the Examples.

FIG. 2B is a chromatogram for a lyophilized human IgG starting material after reconstitution at 200 mg/mL, as further explained in the Examples.

FIG. 2C is a chromatogram for a spray-dried human IgG formulation after reconstitution at 200 mg/mL, as further explained in the Examples.

FIG. 3 shows the percent monomer analysis (SEC-HPLC) of antibody-containing formulations before spray drying, after spray drying and reconstitution with 0.05% w/v Tween-80, and after spray drying and reconstitution with 0.1% w/v Tween-80, as further explained in the Examples. Formulations were reconstituted to provide concentrations of 5 mg/mL. Reconstitution is abbreviated as “recon.” and spray drying as “SD” in this figure.

FIG. 4 shows the percent monomer analysis (SEC-HPLC) of antibody-containing formulations before spray drying, after spray drying and reconstitution with 0.05% w/v Tween, and after spray drying and reconstitution with 0.1% w/v Tween-80 (both at 70 mg/mL and 150 mg/mL), as further explained in the Examples. Reconstitution is abbreviated as “recon.” and spray drying as “SD” in this figure.

FIG. 5A is a chromatogram for a stock antibody composition, as further explained in the Examples.

FIG. 5B is a chromatogram for a reconstituted antibody-containing formulation after reconstitution at 190 mg/mL, as further explained in the Examples.

FIG. 6 shows the percent monomer analysis (SEC-HPLC) of two antibody-containing formulations (each having a different sugar excipient) before spray drying and after spray drying and reconstitution with 0.1% w/v Tween-80, as further explained in the Examples. After spray drying formulations were reconstituted to provide a concentration of 140 mg/mL. Spay drying has been abbreviated as “SD” in this figure.

FIG. 7 shows the percent monomer analysis (SEC-HPLC) of an antibody-containing formulation before spray drying and after spray drying and reconstitution with 0.1% w/v Tween-80, as further explained in the Examples. The after spray drying formulation was reconstituted to provide a concentration of 190 mg/mL. The abbreviation “SD” stands for “spray drying” in this figure.

FIG. 8 shows the percent monomer analysis (SEC-HPLC) of two antibody-containing formulations (each having a different amount of the same sugar excipient) before spray drying and after spray drying and reconstitution with 0.1% w/v Tween-80, as further explained in the Examples. After spray drying formulations were reconstituted to provide a concentration of 190 mg/mL. Spray drying has been abbreviated as “SD” in this figure.

### DETAILED DESCRIPTION OF THE INVENTION

Before describing the present invention in detail, it is to be understood that this invention is not limited to the antibody, diluents, excipients, spray-drying methods, and the like as such may vary. It is also to be understood that the terminology used herein is for describing particular embodiments only, and is not intended to be limiting.

It must be noted that, as used herein, the singular forms “a,” “an,” and “the” include plural referents unless the context clearly dictates otherwise. Thus, for example, reference to “an antibody” includes a single antibody as well as two or more of the same or different antibodies, reference to an excipient refers to a single excipient as well as two or more of the same or different excipients, and the like.

In describing and claiming the present invention, the following terminology will be used in accordance with the definitions described below.

The term “amino acid” refers to any molecule containing both an amino group and a carboxylic acid group. Although the amino group most commonly occurs at the beta position (i.e., the second atom from the carboxyl group, not counting the carbon of the carboxyl group) to the carboxyl function, the amino group can be positioned at any location within the molecule. The amino acid can also contain additional functional groups, such as amino, thio, carboxyl, carboxamide, imidazole, and so forth. As used herein, the term “amino acid” specifically includes amino acids as well as derivatives thereof such as, without limitation, norvaline, 2-aminoheptanoic acid, and norleucine. The amino acid may be synthetic or naturally occurring, and may be used in either its racemic or optically active (D-, or L-) forms, including various ratios of stereoisomers. The amino acid can be any combination of such compounds. Most preferred are the naturally occurring amino acids. The naturally occurring amino acids are phenylalanine, leucine, isoleucine, methionine, valine, serine, proline, threonine, alanine, tyrosine, histidine, glutamine, asparagines, lysine, aspartic acid, glutamic acid, cysteine, tryptophan, arginine, and glycine.

By “oligopeptide” is meant any polymer in which the monomers are amino acids totaling generally less than about 100 amino acids, preferably less than 25 amino acids. The term oligopeptide also encompasses polymers composed of two amino acids joined by a single amide bond as well as polymers composed of three amino acids.

“Dry” when referring to a powder (e.g., as in “dry powder”) is defined as containing less than about 10% moisture. Preferred compositions contain less than 7% moisture, more preferably less than 5% moisture, even more preferably less than 3% moisture, and most preferably less than 2% moisture. The moisture of any given composition can be determined by, for example, the Karl Fischer titrimetric technique using a Mitsubishi moisture meter model # CA-06.

As used herein, an “excipient” is an intended, nonantibody and nondiluent component of a particle, powder or composition. Thus, “excipients” such as buffers, sugars, amino acids, and so forth are intended components of a formulation and stand in contrast to unintended components of a formulation such as impurities (e.g., dirt) and the like.

A “therapeutically effective amount” is the amount of the antibody required to provide a desired therapeutic effect. The exact amount required will vary from subject to subject and will otherwise be influenced by a number of factors, as will be explained in further detail below. An appropriate “therapeutically effective amount,” however, in any individual case can be determined by one of ordinary skill in the art.

The term “substantially” refers to a system in which greater than 50%, more preferably greater than 85%, still more preferably greater than 92%, and most preferably greater than 96%, of the stated condition is satisfied.

The term “antibody” refers to an immunoglobulin protein that is capable of binding another molecule, typically referred to as an “antigen.” As used herein, the term “antibody” shall be understood to include an entire antibody as well as any fragment thereof (e.g., Fab, F(ab)₂, Fv, single polypeptide chain binding molecule [as described in, for example, U.S. Pat. No. 5,260,203] and so forth) that is capable of binding the antigen. In addition, the term “antibody” shall encompass all antibody types, e.g., polyclonal, monoclonal, and those produced by the phage display techniques, as well as all antibody classes, subclasses, subtypes, and so forth, including, for example, IgG (including subclasses IgG₁, IgG₂, IgG₃, and IgG₄), IgM (including subclasses IgM₁ and IgM₂), IgA (including subclasses IgA₁ and IgA₂), IgD, and IgE.

The term “patient,” refers to a living organism suffering from or prone to a condition that can be prevented or treated by administration of an antibody or antibody fragment, and includes both humans an animals.

“Optional” and “optionally” means that the subsequently described circumstance may or may not occur, so that the description includes instances where the circumstance occurs and instances where it does not. Thus, for example, a reconstituted composition comprising an “optional excipient” includes reconstituted compositions comprising one or more excipients as well as reconstituted compositions lacking any excipient.

Turning to a first embodiment then, the invention provides a composition comprising antibody-containing particles, wherein the particles have a certain size. It has been found that powders comprised of particles having a mass median diameter of greater than 7.5 μm and less than about 500 μm formed reconstituted compositions in a facile manner. Typically, however, the particles have a mass median diameter of greater than 7.5 μm and less than about 100 μm. Larger particles may retain undesired amounts of moisture and may reconstitute relatively slowly. Smaller particles may require more stringent procedures for their manufacture and/or additional processing steps such as comminution. Thus, while particles falling outside of the ranges provided herein can be used in accordance with the present invention, particles within this range are preferred. A plurality of antibody-containing particles as described herein conveniently forms a powder.

It is particularly preferred, however, that the particles have a mass median diameter of greater than 10 μ/m to less than about 100 μm, more preferably from greater than 10 μm to less than about 50 μm, still more preferably greater than 10 μm to less than about 30 μm, with a mass median diameter of greater than about 15 μm to less than about 30 μm being most preferred.

Particles having a desired size range can be provided through any number of methods. For example, relatively large antibody-containing particles can micronized to a suitable size via milling. Commercially available mills, such as STOKES® mills from DT Industries (Bristol, Pa.) can be used to reduce relatively large particles into smaller particles having the desired size. A number of mill types can be used and include, for example, air-jet mills and mills comprising moving internal parts such as plates, blades, hammers, balls, pebbles, and so on, which are used to crush or otherwise render undesired larger particles into smaller particles of a desired size.

To ensure that the particles have the desired size, the particles can be analyzed using known techniques for determining particle size. For example, the particles can be visually inspected and/or passed through one or more mesh screens having openings of a known size. With respect to visual inspection, microscopy techniques including optical, scanning electron microscopy (SEM), and transmission electron microscopy techniques can be used. In addition, particle size analysis can take place using laser diffraction methods. Commercially available systems for carrying out particle size analysis by laser diffraction are available from Clausthal-Zellerfeld (HELOS H1006).

With respect to measuring the particle size, any number of techniques can be used. For example, the mass median diameter of a powder can be measured using a Horiba CAPA-700 particle size analyzer (Horiba Instruments Inc., Irvine Calif.) or similar instrument. Particle size measurements are generally based upon centrifugal sedimentation of dispersed particles in a suspending medium. The mass median diameter, which is based on the particle's Stokes' diameter, can be calculated using the particle density and the density and viscosity of the suspending medium.

The antibody-containing particles can take any shape, and the invention is not limited in this regard. Exemplary particle shapes include spheroidal, oblong, polygonal, ringed, and so forth. Regardless of its morphology, an antibody-containing particle has an antibody within the particle. This is in contrast to “antibody-attached particles” in which an antibody is attached, typically covalently, to the surface of a bead, resin, or similar substrate, commonly used in, for example, antibody-based detection assays. Thus, the term “antibody-containing particles” specifically excludes such “antibody-attached particles.”

The antibody-containing particles (typically in the form of a powder) can be prepared in a number of different approaches, including, for example, forming a spray-dried powder, comminuting a freeze-dried product, and others.

Spray drying an antibody-containing liquid represents a preferred approach for providing antibody-containing particles. Spray drying can be performed as described generally in the “Spray Drying Handbook”, 5^{th} ed., K. Masters, John Wiley & Sons, Inc., NY, N.Y. (1991), and in Platz, R., et al., International Patent Publication Nos. WO 97/41833 and WO 96/32149.

In brief, the spray drying process for the present purposes begins by providing an antibody-containing liquid. Typically, although not necessarily, the antibody-containing liquid is in the form of an aqueous solution or suspension, depending on the solubility of the antibody, the amount of the antibody, and the pH of the medium. Thus, the antibody is generally first dissolved or suspended in water, optionally comprising a pH adjusting agent (e.g., an acid or base) and/or a buffer. As used herein, the antibody-containing liquid to be spray dried is interchangeably referred to as the “feed liquid” or “antibody-containing feed liquid.”

The typically aqueous feed liquid generally has a pH in the range of from about 3 to about 11, more typically between from about 3.5 to about 9, with more neutral pHs (e.g., from about 5.5 to about 7.8) being most preferred. Thus, the feed liquid can have a pH ranging from about 3 to about 4, from about 4 to about 5, from about 5 to about 6, from about 6 to about 7, from about 7 to about 8, or from about 8 to about 9. Adjustments to the pH of the feed liquid can be accomplished by adding an acid or base.

The feed liquid can optionally contain one or more additional excipients. Nonlimiting examples of excipients that can be added to the feed liquid include a water-miscible solvent, amino acids, amino acid derivatives, oligopeptides, carbohydrates, inorganic salts, antimicrobial agents, antioxidants, surfactants, buffers, acids, bases, and combinations thereof.

Optionally, one or more water-miscible solvents can be included in the feed liquid. For example, a water-miscible solvent such as acetone, an alcohol and other known water-miscible solvents can be added to the feed liquid. Representative alcohols are lower alcohols such as methanol, ethanol, propanol, isopropanol, and so forth. When an aqueous feed liquid comprises a water-miscible solvent, a mixed solvent system is formed and will typically contain from about 0.1% to about 80% of the water miscible solvent, more preferably from about 20% to about 40%, and most preferably from about 10 to about 30% of the water miscible solvent.

The feed liquid can also optionally comprise one or more amino acids. Exemplary amino acids (and derivatives thereof) include those selected from the group consisting of glycine, alanine, valine, asparagine, leucine, norleucine, isoleucine, phenylalanine, tryptophan, tyrosine, proline, methionine, acylated forms thereof, and combinations thereof. Preferably, however, the amino is histidine, leucine, or a combination thereof.

Oligopeptides comprising any of the herein described amino acids are also suitable for use as an optional excipient in the feed liquid. Preferred oligopeptides, however, include poly-lysine (comprising, for example, 2 to 10 lysine residues, more preferably 4 to 10 lysine residues), poly-glutamic acid (comprising, for example, 2 to 10 glutamic acid residues, more preferably 4 to 10 lysine residues), and poly-lysine/alanine (comprising, for example, 2 to 5 residues of lysine and alanine in any sequential order), dileucine, leu-leu-gly, leu-leu-ala, leu-leu-val, leu-leu-leu, leu-leu-ile, leu-leu-met, leu-leu-pro, leu-leu-phe, leu-leu-trp, leu-leu-ser, leu-leu-thr, leu-leu-cys, leu-leu-tyr, leu-leu-asp, leu-leu-glu, leu-leu-lys, leu-leu-arg, leu-leu-his, leu-leu-nor, gly-leu-leu, ala-leu-leu, val-leu-leu, ile-leu-leu, met-leu-leu, pro-leu-leu, phe-leu-leu, trp-leu-leu, ser-leu-leu, thr-leu-leu, cys-leu-leu, tyr-leu-leu, asp-leu-leu, glu-leu-leu, lys-leu-leu, arg-leu-leu, his-leu-leu, nor-leu-leu, leu-gly-leu, leu-ala-leu, leu-val-leu, leu-ile-leu, leu-met-leu, leu-pro-leu, leu-phe-leu, leu-trp-leu, leu-ser-leu, leu-thr-leu, leu-cys-leu, leu-try-leu, leu-asp-leu, leu-glu-leu, leu-lys-leu, leu-arg-leu, leu-his-leu, leu-nor-leu, lys-lys-lys, and combinations thereof. A particularly preferred oligopeptide is leu-leu-leu or “trileucine.”

A carbohydrate such as a sugar, a derivatized sugar such as an alditol, aldonic acid, an esterified sugar, and a sugar polymer can be present as an optional excipient in the feed liquid. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. Preferred carbohydrates for use in the feed liquid include sucrose and trehalose. In some circumstances, it is preferred that the feed liquid, as well as the resulting particles and powder, does not contain melezitose.

The optional excipient in the feed liquid can also include an inorganic salt or buffer such as citric acid, sodium chloride, potassium chloride, sodium sulfate, potassium nitrate, sodium phosphate monobasic, sodium phosphate dibasic, and combinations thereof. Salts that provide monovalent or divalent cations such as sodium, potassium, aluminum, manganese, calcium, zinc, and magnesium are preferred. Preferably, the salt or buffer is selected from the group consisting of citric acid, sodium phosphate monobasic, sodium phosphate dibasic, and combinations thereof.

The feed liquid can also optionally include an antimicrobial agent for preventing or deterring microbial growth in feed liquid, thereby being present in the resulting formulation. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

Optionally, an antioxidant can be present in the feed liquid as well. Antioxidants are used to prevent oxidation, thereby preventing the deterioration of the antibody. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

The feed liquid can also optionally comprise a surfactant. Exemplary surfactants include: polysorbates such as Tweens, e.g., “Tween-20” and “Tween-80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations. A particularly preferred surfactant is Tween-20, Tween-80 or a combination thereof.

Acids or bases may be present as an optional excipient in the feed liquid. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

Other optional excipients suitable for use in the compositions according to the invention are listed in “Remington: The Science & Practice of Pharmacy,” 19^{th} ed., Williams & Williams, (1995), “Physician's Desk Reference, 52 ed., Medical Economics, Montvale, N.J. (1998), WO 96/32096, and in “Handbook of Pharmaceutical Excipients,” 3^{rd} ed., Kibbe, A.H. Editor (2000).

In order to provide antibody-containing particles that contain a relatively large amount of antibodies per particle, a relatively high concentration of antibody will be present in the feed liquid. The concentration of the antibody and optional excipient(s) (e.g., sugar, carrier, salt, and so forth) in the feed liquid is conventionally referred to as the “solids concentration.” Essentially, the solids concentration represents the total concentration of all components present in the antibody-containing feed liquid that are ultimately retained in the resulting spray-dried particles. When present, however, volatile salts (such as NaHCO₃) are included as part of the solids concentrations even though such salts may not actually be present in the spray-dried particles.

Exemplary solids concentrations in the feed liquid include concentrations from about 0.01% (weight/volume or “w/v”) to about 30% (w/v), from about 0.5% (w/v) to about 30% (w/v), and from about 1.0% (w/v) to about 20% (w/v), although solids concentrations outside of this range can also be used. In terms of mg/ml, then, corresponding exemplary solids concentration are from about 0.1 mg/ml to about 300 mg/ml, from about 5 mg/ml to about 300 mg/ml, and from about 10 mg/ml to about 200 mg/ml. Specifically, the feed liquid will typically possess one of the following solids concentrations: 0.1 mg/ml or greater, 5 mg/ml or greater, 7.5 mg/ml or greater, 10 mg/ml or greater, 15 mg/ml or greater, 20 mg/ml or greater, 30 mg/ml or greater, 40 mg/ml or greater, or 50 mg/ml or greater. Preferably, feed liquids have a solids concentration of greater than 7.5 mg/ml or greater, more preferably from about 10 to about 15 mg/ml. In addition, solid concentrations of 100 mg/mL can also be used. Typically, although not necessarily, the antibody will constitute greater than about 50%, more preferably greater than about 80%, more preferably greater than about 90%, still more preferably greater than about 95%, yet still more preferably greater than about 98%, and most preferably greater than about 99%, of the total solids concentrations.

It is preferable to spray dry the feed liquid at the higher ends (i.e., higher solids content) of the preferred solids concentration ranges, since higher solids concentrations typically correspond to a relatively higher concentration of the antibody within any given particle. In this way, a smaller number of antibody-containing particles, and, by extension, powder, is required to provide a reconstituted formulation having a desired antibody concentration. In addition, relatively higher concentrations of an antibody in the feed liquid results in a relatively lower ratio of “exposed-to-internal” amounts of antibodies in a droplet surface during spray drying, thereby decreasing the relative amount of degradation associated with the air-droplet interface as relatively more antibody is completely located within the droplet.

Ultimately, the amount of the antibody in the spray-dried particles will typically contain at least about one of the following percentages of antibody: 1%, 5%, 10%, 20%, 30%, 40%, 50%, 60%, 70%, 80%, 90% or more by weight. Preferably, the powder formed from the antibody-containing particles (and after one or more optional excipients are added) will contain a total amount of least about 50%, e.g., from about 50 to 99.9% by weight, of antibody-containing particles.

The amount of any individual excipient (when present) in the reconstituted composition or in spray-dried powder will vary depending on the activity of the excipient and particular requirements of the desired spray-dried powder and/or reconstituted composition. Typically, the optimal amount of any individual excipient is determined through routine experimentation, i.e., by preparing compositions containing varying amounts of the excipient (ranging from low to high), examining the stability, reconstitutability (including reconstitution time), aggregate percentage in the formulation, and so forth, and then further determining the range at which optimal performance is attained with no significant adverse effects.

Generally, however, the excipient will be present in the reconstituted composition or spray-dried powder in an amount of from about 0.01% to about 99% by weight, preferably from about 5% to about 98% by weight, more preferably from about 15% to about 95% by weight of the excipient, with concentrations less than 30% by weight most preferred.

Once the antibody and optional excipient(s) have been selected, the antibody and optional excipient(s) are added to a liquid to form a feed liquid for spray drying. Although the liquid is generally aqueous, any liquid suitable for spray drying can be used. Generally, the feed liquid will be a solution, e.g., aqueous solution, although suspensions can also be used. The feed liquid is typically mixed well prior to spray drying.

The feed liquid is then spray dried in a conventional spray drier, such as those available from commercial suppliers such as Niro A/S (Denmark), Buchi (Switzerland) and the like, resulting in a dry powder. Optimal conditions for spray drying the solutions will vary depending upon the formulation components, and are generally determined experimentally. The gas used to spray dry the material is typically air, although inert gases such as nitrogen or argon are also suitable. Moreover, the temperature of both the inlet and outlet of the gas used to dry the sprayed material is such that it does not cause decomposition of the active agent in the sprayed material. Such temperatures are typically determined experimentally, although generally, the inlet temperature will range from about 50° C. to about 200° C., while the outlet temperature will generally range from about 30° C. to about 150° C. Preferred parameters include atomization pressures ranging from about 20 to 150 psi (0.14 to 1.03 MPa), and preferably from about 30 to about 40 to 100 psi (0.21-0.28 to 0.69 MPa). Typically the atomization pressure employed will be one of the following: 20 psi (0.14 MPa), 30 psi (0.21 MPa), 40 psi (0.28 MPa), 50 psi (0.34 MPa), 60 psi (0.41 MPa), 70 psi (0.48 MPa), 80 psi (0.55 MPa), 90 psi (0.62 MPa), 100 psi (0.69 MPa), 110 psi (0.76 MPa), 120 psi (0.83 MPa) or above. Spray-dried particles are physically distinct from powders prepared by other drying methods, and typically exhibit morphologies and thermal histories (including glass transition temperatures, glass transition widths, and enthalpic relaxation profiles) that differ from those of powders prepared by other drying methods such as lyophilization.

The spray-dried powder will generally have a moisture content below about 20% by weight, usually below about 10% by weight, and preferably below about 6% by weight. More preferably, the spray-dried powder will typically possess a residual moisture content below about 3%, more preferably below about 2%, and most preferably between about 0.5 and 2% by weight. Such low moisture-containing solids tend to exhibit a greater stability upon packaging and storage. Optionally, the spray-dried powder can be stored in sealed containers such as blister packages, vials, and the like, to prevent hygroscopic growth.

Comminuting a freeze-dried or lyophilized product containing antibodies represents another approach for providing antibody-containing particles. In this approach, antibodies are introduced into water to form a mixture. Optionally, one or more excipients (e.g., sugars such as sucrose and trehalose, bulking agents such as mannitol, surfactants, antioxidants, and so forth) as described above with respect to spray drying can also be introduced into the mixture. Once the mixture is formed, the mixture's temperature is reduced to below its eutectic point using conventional techniques. Water from the mixture is then sublimed, thereby forming a freeze-dried product.

Conveniently, commercially available freeze dryers are available for carrying out the freezing and subliming steps. Examples of commercially available freeze dryers include those available from Hull Company (Warminster, Pa.) and Steris Corporation (Mentor, Ohio). Regardless of the specific freeze-drying technique used, the result of freeze-drying is the formation of a freeze-dried product in the form of a “dry foam” or “cake.”

Subsequent comminution of the lyophilized product results in antibody-containing particles. Comminution of the lyophilized product can take place using any number of art-known methods. As stated previously with respect to reducing particle size generally, commercially available mills are available for comminuting particles into a desired particle size. Particles prepared from comminuting freeze-dried materials are, however, different from particles prepared from spray-drying techniques. For purposes of the present invention, spray-drying techniques along with the resulting spray-dried particles are preferred.

Other approaches for forming antibody-containing particles can also be used. For example, granulation techniques, precipitation techniques, and so forth can be used to prepare particles. If necessary, a comminution step (as discussed above) can be carried out in order to provide antibody-containing particles having the desired size.

No matter which approach is used to provide the antibody-containing particles, the antibody-containing particles are recovered and combined together, thereby forming a powder. It is preferred that the antibody-containing particles are maintained under dry (i.e., relatively low humidity) conditions. Moreover, to the greatest extent possible, further handling (e.g., processing, packaging, and storage) of the particles and powder is conducted under dry conditions.

If desired, one or more particulate excipients can be added to the powder. In this embodiment, the powder will comprise not only antibody-containing particles (that may also contain one or more excipients) but separate particles of an excipient as well. Stated differently, the powder can comprise a mixture of physically separate “excipient only” particles in addition to the antibody-containing particles. One or more of the above-identified excipients can be added to the powder so long as the excipient can be provided in particulate form.

Optionally, the powder can be divided into portions. For example, the powder can be divided based on weight, and stored in, for example, a vial or a syringe. Optimally for therapeutic applications, each divided portion will contain a unit dose of the antibody. Advantageously, a kit can be provided wherein the powder can be packaged in a vial (e.g., a glass or plastic vial) along with instructions for using the powder. The kit may optionally include a vial of premeasured diluent for use in reconstituting the powder. In addition, the kit optionally includes a needle and syringe for administering the reconstituted powder to a patient.

Advantageously, the antibody-containing particles (typically in the form of a powder) can be reconstituted to form a reconstituted composition. The antibody is present in the reconstituted composition at a concentration suitable for administration to a patient. Preferably, however, the reconstituted composition comprises an antibody in an amount of from about 25 mg/mL to about 1000 mg/mL, a diluent and an optional excipient, wherein the reconstituted composition is formed from a powder (typically a spray-dried powder) comprised of the antibody and the optional excipient. Typically, although not necessarily, the powder is in the form of a powder prepared by a spray drying process (sometimes referred to as a spray-dried powder).

The reconstituted composition is typically prepared by following the method comprising the step of providing a powder (typically a spray-dried powder) comprised of an antibody and adding a diluent in order to form the reconstituted composition, wherein the antibody is present in the reconstituted composition in an amount of from about 25 mg/mL to about 1000 mg/mL. The step of adding the diluent in order to reconstitute the powder typically, although not necessarily, takes place at room temperature.

Any diluent suitable for reconstituting compositions can be used and the invention is not limited in this regard. Preferred diluents, however, are those selected from the group consisting of bacteriostatic water for injection, dextrose 5% in water, phosphate-buffered saline, Ringer's solution, saline, sterile water, deionized water, and combinations thereof.

The amount of the diluent added to the powder is an amount such that the resulting concentration is suited to the intended application. Those of ordinary skill in the art know or can experimentally determine an appropriate antibody concentration for any given application. Typically, however, the concentration of the antibody in the reconstituted composition is about 1000 mg/mL or less. Thus, for example, completely spray drying a feed liquid comprising 1000 mg of an antibody and completely recovering the entire spray-dried powder will require 1 mL of diluent to form a reconstituted composition having an antibody concentration of 1000 mg/mL, 2 mL of diluent to form a reconstituted composition having an antibody concentration of 500 mg/mL, and so forth.

For subcutaneous administration, a preferred concentration range of the antibody in the reconstituted composition is from about 25 mg/mL to about 750 mg/mL, more preferably from about 25 mg/mL to about 500 mg/mL, still more preferably from about 50 mg/mL to about 450 mg/mL, yet still more preferably from about 70 mg/mL to about 400 mg/mL, and still more preferably from about 100 mg/mL to about 300 mg/mL. Another preferred range of the antibody is from about 25 mg/mL to about 250 mg/mL.

With respect to intravenous administration, exemplary antibody concentrations include from about 2.5 mg/mL to about 100 mg/mL, from about 5 mg/mL to about 75 mg/mL, and from about 10 mg/mL to about 50 mg/mL.

In some instances, the antibody concentration in the reconstituted composition will be higher than that in the solution (e.g., feed liquid for spray-dried powders) used to form the antibody-containing particles. For example, the antibody concentration in the reconstituted formulation can be about 2 to about 50 times, preferably about 3 to about 25 times, and more preferably about 4 to about 10 times, than that used in the solution (e.g., feed liquid for spray-dried powders) used to form the antibody-containing particles.

In some circumstances, the reconstituted formulation has been prepared from a feed liquid of the antibody and an excipient that prevents or reduces chemical or physical instability of the antibody upon spray drying and subsequent storage (e.g., a carbohydrate and/or amino acid). Exemplary molar ratios of the excipient to antibody include: about 0.0001 to 0.001 mole excipient to 1 mole antibody; about 0.001 to 0.01 mole excipient to 1 mole antibody; about 0.01 to 0.1 mole excipient to 1 mole antibody; about 0.1 to 1 mole excipient to 1 mole antibody; about 1 to 10 mole excipient to 1 mole; about 10 to 100 mole excipient to 1 mole antibody; and about 100 to 1000 mole excipient to 1 mole antibody.

The reconstituted composition preferably has substantially no aggregates. It is preferred that the particles, powders, and reconstituted compositions have less than 20% by weight total aggregates, more preferably less than 10% by weight total aggregates, still more preferably less than 5% by weight total aggregates, still yet more preferably less than 2% by weight total aggregates, with less than 1% by weight total aggregates being most preferred.

The time required to reconstitute the powder will depend on a variety of factors including the antibody, the presence and effect of one or more optional excipients, the diluent used, and so forth. The reconstituted compositions, however, preferably become visually clear within about 15 minutes, more preferably within about 10 minutes, and most preferably within about 5 minutes, of adding the diluent.

As previously stated, the reconstituted composition optionally comprises an excipient. The excipient in the reconstituted composition can be present by virtue of its presence in the antibody-containing particles that make up the powder. In addition, the excipient can be present in the composition as a result of being added subsequent to the formation of the antibody-containing particles forming the powder, but prior to reconstitution. Furthermore, the excipient can be present in the reconstituted composition by having been added with or following the addition of the diluent. Again, any excipient commonly used in pharmaceutical compositions may be used and can include any previously discussed excipients such as, for example, those selected from the group consisting of amino acids, amino acid derivatives, oligopeptides, carbohydrates, inorganic salts, antimicrobial agents, antioxidants, surfactants, buffers, acids, bases, and combinations thereof.

Preferably, the reconstituted composition is suited for injection. Thus, it is preferred that the formulations described herein—both prior to and after reconstitution—are free from bacteria and free from bacterial endotoxins. In addition, the reconstituted formulations preferably meet or exceed customary injectable particulate level requirements wherein there are 3000 or less particles of a size 10 μm or greater when determined by light microscopy per container (6000 or less when determined by light obscuration) and 300 or less particles of a size 25 μm or less when determined by light microscopy per container (600 or less when determined by light obscuration). These requirements are in contrast to the requirements of other routes of administration, such as the pulmonary route. In the pulmonary route, for example, commonly acceptable inhalable powder requirements provide that bacteria can be present up to about 10 colony-forming units (CFU) per gram.

Sterility can be assured by, for example, carrying out the process used to provide the antibody-containing particles (e.g., spray-drying process) as well as subsequent packaging under completely aseptic conditions. In addition, sterilization can be accomplished by irradiation as well as via chemical means (e.g., exposing the final composition to vaporized hydrogen peroxide). Moreover, a combination of techniques can be used. With respect to antibody-containing formulations that have been spray dried, the spray drying can be conducted in an aseptic closed system wherein incoming solution and air streams are filtered to ensure sterility. Thus, for example, 0.2 μ/m filters can be used to filter the feed liquid. In addition, a 0.2 μm filter can be used to filter the gas used in the spray-drying process.

Once prepared, the antibody-containing powder is filled into a suitable container (e.g., a glass vial), again under aseptic conditions. Any mechanical filler can be used to fill the desired container and the invention is not limited in this regard. Exemplary fillers are available from M&O Perry Industries (Corona, Calif.) and include their Model 2115 filler. Such fillers are capable of filling at least 35 to 45 of the desired containers per minute at fill weights of about 100-200 mg. Once the powders are packaged with air-tight seals, the packaged powders can be transported and distributed and stored until needed.

With respect to the antibody, any antibody can be used in the antibody-containing particles as well as the compositions described herein. Nonlimiting examples of antibodies useful in accordance with the invention include antibodies to microorganisms (including respiratory pathogens), monoclonal antibodies directed against tumor antigens and antibodies to cell receptors (including receptors involved in inflammation and allergy). As full-length antibodies as well as antibody-fragments have demonstrated value as therapeutic agents, diagnostic agents, and/or detection agents, the reconstituted compositions may comprise either a full-length antibody or an antibody fragment. When an antibody fragment is used, any fragment type may be used so long as the antibody fragment of interest has value, e.g., value as a therapeutic agent, diagnostic agent, detection agent, and so forth. Generally, however, the antibody fragment will usually be selected from the group consisting of Fab fragments, F(ab)₂ fragments, Fv fragments, and single polypeptide chain binding molecules. Immunoconjugates wherein the antibody is attached (generally, although not necessarily, covalently attached) to a therapeutic or diagnostic agent such as a radioactive pharmaceutical, chemotherapeutic agent or a radioactive label are also envisioned. Pharmaceutically acceptable salts of any of the above may also be used. The antibody can also be formulated with lipids, liposomes, microspheres and the like.

Antibodies suitable for use in the compositions of this invention include IgA, IgE, IgG, IgD and IgM. It is preferred, however, that IgA, IgG and IgM are used, with IgG and IgA antibodies being particularly preferred.

The antibody used herein can be obtained using techniques known to those of ordinary skill in the art. Such techniques include, for example, recombinant techniques, peptide synthetic techniques, and isolation techniques.

For example, polyclonal antibodies can be prepared by injecting (e.g., by subcutaneous, intraperitoneal, or intramuscular injection) into an animal host the antigen against which the antibody will bind. The animal host is typically, although not necessarily, a rabbit or a mouse. Often, the injection site on the host will be shaved and swabbed with alcohol prior to the injection. The injection generally occurs in multiple sites in the animal host. Typically, the total volume of the antigen-containing injection is not more than about 1 mL.

Having injected the antigen into the host animal, the host animal's immune response is allowed to start producing antibodies directed against the antigen. Specifically, lymphocytes of the host animal will produce and secrete antibodies to the antigen into the blood stream. Although each binding to same antigen, the different antibodies likely bind to different antigenic determinants (referred to as “epitopes”), thereby providing the “polyclonal” nature of antibodies produced in this approach.

In order can recover the polyclonal antibodies now circulating in the animal host's blood stream, blood collection from the animal is performed. The blood can be collected using conventional techniques such as inserting the tip of a needle equipped with a syringe into the host. Blood is then collected and typically allowed to clot at 37° C. overnight. The clotted blood is then generally refrigerated for 24 hours before the serum is recovered by conventional techniques (e.g., by running a centrifuge at 2500 revolutions per minute for about 20 minutes and collecting the antibody-containing portion). Blood collection is performed periodically, such as at about four weeks following injection of the antigen, seven weeks following injection of the antigen, 11 weeks following injection of the antigen, and every three weeks thereafter.

The blood collections serve the dual purposes of determining the titer of the desired antibody (through, for example, conventional enzyme-linked immunosorbant assay or “ELISA”) as well as recovering the antibodies (assuming a sufficient titer is present). The antibodies in any given sample can be recovered through, for example, centrifuging, separating through an affinity column (e.g., a “protein-A” column), and a combination thereof. Additional recovery techniques are known to those of ordinary skill in the art and can be used as well.

To the extent that any given sample of the blood has an insufficient or a generally low titer, a number of approaches are used to increase the titer and/or maintain the titer at levels sufficient to provide antibodies. In one approach, the antigen introduced into the animal host can be conjugated to a protein (e.g., keyhole limpet hemocyanin or serum albumin), thereby increasing the overall antigenicity of the antigen. In addition, other substances known as adjuvants can be injected along with the antigen in order to enhance the immunogenic response. Typically, complete Freund's adjuvant is injected along with the antigen in the initial injection and incomplete Fruend's adjuvant is injected along with the antigen during subsequent injections. Both complete Freund's adjuvant and incomplete Freund's adjuvant are available commercially and through, for example, Sigma-Aldrich, Inc. (St. Louis, Mo.).

Monoclonal antibodies can also be used in accordance with the present invention. Produced from a cultured colony of cells derived from a single lymphocyte, monoclonal antibodies recognize only one eptitope on an antigen. Monoclonal antibodies can conveniently be prepared using the process described in Kohler et al. (1975) Nature 256:495.

Briefly, monoclonal antibodies can be prepared by first injecting the antigen of interest into a suitable animal host such as a mouse. Thereafter, the animal host is euthanized and the spleen is removed so as to recover the animal host's antibody-producing lymphocytes in the spleen. Due to their limited growth potential, the normal, antibody-producing lymphocytes are fused with cancer cells in order to take advantage of the prolific and virtually unlimited growth of cancer cells. The fusion of the lymphocyte and cancer cell results in a hybridoma cell. When placed in a suitable cell medium, the hybridoma cell line can grow indefinitely. Fusion of the two different types of cells occurs using a conventional fusing agent, such as polyethylene glycol.

The cancer cell used in the hybridoma and the cell culture medium are specifically chosen so that it is possible to select for hybridomas. This can be accomplished by using a myeloma cell that has lost the ability to synthesize hypoxanthine-guanine phosphoribosyltransferase (HGPRT) as the cancer cell and a HAT medium (i.e., a cell culture medium comprising hypoxanthine, aminopterin and the pyrimidine thymidine) as the cell culture medium. This approach is premised on the ability of cells to obtain life-sustaining purines through two pathways: a first pathway that requires the enzyme HGPRT in the presence of hypoxanthine; and a second pathway mediated by folic acid that is blocked in the presence of a folic acid antagonist such as methotrexate or aminopterin. The logic of this approach is as follows: (i) unfused myeloma cells lacking HGPRT will not grow because they cannot use the hypoxanthine present in the HAT medium since they lack the necessary enzyme HGPRT and because the folic acid antagonist, aminopterin, in the HAT medium blocks the folic acid mediated pathway to purine synthesis; (ii) unfused lymphocytes cannot grow indefinitely due to their limited life spans; and (iii) hybridoma cells (successful fusions between the lymphocyte and cancer cells) are able to growth indefinitely because the lymphocyte provides the HGPRT necessary to utilize the hypoxanthine necessary to form purines.

Preferred HGPRT-deficient cells include the murine-based MOPC-21 and MPC-11 cells available from the Salk Institute Cell Distribution Center (San Diego, Calif.) and the SP2 cells available from the American Type Culture Collection (Rockville, Md.). Cell media, including the HAT medium, are available commercially from sources such as Sigma-Aldrich, Inc. (St. Louis, Mo.).

The hybridomas are then assayed for the production of antibodies using conventional techniques such as immunoprecipitation, radioimmunoassay, ELISA or a similar technique. When a hybridoma is identified that produces the desired antibody (i.e., an antibody directed against a specific epitope on a specific antigen), the hybridoma is then subcloned by placing the hybridoma in a suitable medium and allowed to grow. In this way, a monoclonal population is formed.

The monoclonal antibodies secreted by subcloned hybridomas are separated using conventional techniques such as through protein-A columns, gel electrophoresis, the affinity chromatography, and the like.

In addition, the antibodies can be derived using recombinant DNA technology. For example, the DNA encoding the monoclonal antibodies can be isolated from the hybridoma cells through, for example, use of the appropriate oligonucleotide probes. Thereafter, the DNA can be placed into suitable expression vectors, which can then be transfected into host cells such as E. coli cells, Chinese hamster ovary (CHO) cells or other cell that does not produce immunoglobulins. The DNA obtained from the hybridoma cells can, of course, be modified prior to transfection. For example, the coding sequences for human heavy- and light-chain constant domains or other regions can be substituted for the homologous host (murine) cell's sequences. In this way, the resulting antibody is more humanized and will typically be less antigenic upon administration to a human. See, for example, U.S. Pat. No. 4,816,567.

Also, antibodies can conveniently be obtained through a variety of suppliers. For example, cells producing antibodies can be obtained from the Salk Institute Cell Distribution Center (San Diego, Calif.) and the American Type Culture Collection (Rockville, Md.). A preferred cell line is designated as American Type Culture Collection designated as Patent Deposit Number PTA-4112. The antibodies produced by this cell line are specific for poly(ethylene glycol) and are described in more detail in U.S. Patent Application Publication US 2003/0017504. Additional antibodies specific for poly(ethylene glycol) have been deposited and designated as CCTCC-V-200001 and are described in more detail in U.S. Pat. No. 6,956,849. In addition, commercial suppliers such as Sigma-Aldrich (St. Louis, Mo.) and others can provide antibodies.

The antibody can be adapted or further modified, depending on the needs or desires of the scientist, clinician, or diagnostician. For example, chimeric forms of an antibody that combine two or more portions derived from or based on different organisms can be used. In addition, CDR-grafted antibodies and/or different glycosylated forms can be used. Moreover, antibody-conjugates and antibody fragment-conjugates in which a drug (e.g., chemotherapeutic agent) is bound directly to the non-binding portion of the antibody or antibody fragment can be used.

Any type of antibody can be used and the invention is not limited in this regard. For example, chimeric antibodies can be used in which the whole of the variable regions of a mouse or other host are expressed along with human constant regions, thereby providing the antibody with human effector functions as well as decreased immunogenicity. In addition, humanized and CDR grafted antibodies in which the complimentarity determining regions from the mouse or host antibody V-regions are combined with framework regions from human V-regions, thereby resulting in decreased immunogenicity. In addition, fully human antibodies can be used that have been prepared from synthetic phage libraries or from transgenic mice or other transgenic animals treated such that they synthesize human immunoglobulin germline gene segments. It will be understood that the term “antibody” as used herein encompasses each of these types of antibodies.

Specific antibodies for use in the present invention include, for example (when known, corresponding brand names are provided in parentheses) the antibodies associated with abciximab (ReoPro®), adalimumab (Humira®), afelimobam (Segard®), alemtuzumab (Campath®), antibody to B-lymphocyte (Lymphostat-B™), atlizumab, basiliximab (Simulect®), bevacizumab (Avastin®), biciromab, CAT-213 or bertilimumab, CDP-571 (Humicade™), CDP-860, CDP-870, cetuximab (Erbitux®), clenoliximab, daclizumab (Zenapax®), eculizumab (Alexion™), edrecolomab (Panorex®), efalizumab (Raptiva™), epratuzumab (LymphoCide®), fontolizumab, gavilimomab, gemtuzumab ozogamicin (Mylotarg®), ibritumomab tiuxetan (Zevalin®), infliximab (Remicade®), inolimomab, keliximab, labetuzumab (CEA-Cide®), lerdelimumab (Trabio®), radiolabeled lym-1 (Oncolym®), metelimumab, mepolizumab, mitumomab, muromonad-CD3 (Orthoclone-OKT3®), nebacumab (Centoxin®), natalizumab (Antegren®), odulimomab (Antilfa®), omalizumab (Xolair®), oregovomab (OvaRex®), palivizumab (Synagis®), pemtumomab, pexelizumab, rituximab (Rituxan®), satumomab pendetide (Oncoscint®), sevirumab (Protovir®), siplizumab, tositumomab and I¹³¹tositumomab (Bexxar®), trastuzumab (Herceptin®), tuvirumab, and visilizumab (Nuvion®).

The invention also provides a method of administering a composition to a patient suffering from a condition that is responsive to treatment with an antibody comprising administering, via injection, a therapeutically effective amount of a reconstituted composition comprised of the antibody in an amount (preferably, although not necessarily) of from about 25 mg/mL to about 1000 mg/mL, a diluent and an optional excipient. Typically, the reconstituted composition is formed from a spray-dried powder comprised of the antibody and the optional excipient.

The method of treatment may be used to treat any condition that can be remedied or prevented by administration of the particular antibody. Those of ordinary skill in the art appreciate which conditions a specific antibody can effectively treat. The actual dose to be administered will depend upon the age, weight, and general condition of the subject, as well as the severity of the condition being treated, the judgment of the health care professional, and specific antibody or antibody fragment being used. Therapeutically effective amounts are known to those of ordinary skill in the art and/or are described in the pertinent reference texts and literature. Generally, a therapeutically effective amount will range from about 0.001 mg/kg/day to 100 mg/kg/day, preferably in doses from 0.01 mg/kg/day to 75 mg/kg/day, and more preferably in doses from 0.10 mg/kg/day to 50 mg/kg/day.

The reconstituted compositions can preferably be administered via subcutaneous (sc) injection, intramuscular injection (im), and intravenous (iv) injection (either by infusion or bolus dose), although other forms of injection can also be used. Exemplary unit dosages and routes of administration of the reconstituted compositions designed for therapeutic applications are provided below in Table 1. In the table, a reference to mg/m represents the dose, in mg, per square meter of body surface area (BSA) of the patient. There are several methods for deriving the BSA of patient, including the use of various formulae. An exemplary formula is suggested by Mostellar: BSA in m²=([height of patient in centimeters]×[weight of patient in kilograms]/3600) 2. See Mosteller (1987) N. Engl. J. Med. 22;317(17):1098.

TABLE I
Dosage and Route of Administration for
Various Reconstituted Compositions

| Antibody or Antibody | | | Route of |
|---|---|---|---|
| Fragment | Dose | | Administration |
| Muromonab-CD3 | 5 | mg | iv infusion |
| Abciximab | 7.2 | mg | iv infusion |
| Rituximab | 375 | mg/m² | iv infusion |
| Daclizumab | 80 | mg | iv infusion |
| Basiliximab | 20 | mg | iv infusion |
| Palivizumab | 1200 | mg | im injection |
| Infliximab | 400 | mg | iv infusion |
| Trastuzumab | 160 | mg | iv infusion |
| Gemtuzumab ozogamicin | 9 | mg/m² | iv infusion |
| Alemtuzumab | 30 | mg | iv infusion |
| Ibritumomab tiuxetan | 1.6 | mg | iv injection |

The unit dosage of any given composition can be administered in a variety of dosing schedules depending on the judgment of the clinician, needs of the patient, and so forth. The specific dosing schedule will be known by those of ordinary skill in the art or can be obtained by, for example, reference to the pertinent literature. Exemplary dosing schedules include, without limitation, administration five times a day, four times a day, three times a day, twice daily, once daily, three times weekly, twice weekly, once weekly, twice monthly, once monthly, and any combination thereof. Once the clinical endpoint has been achieved, dosing of the composition is halted.

The following examples are illustrative of the present invention, and are not to be construed as limiting the scope of the invention. Variations and equivalents of these examples will be apparent to those of ordinary skill in the art in light of the present disclosure, the drawings and the claims herein.

All articles, books, patents and other publications referenced herein are hereby incorporated by reference in their entirety.

### Experimental

The practice of the invention will employ, unless otherwise indicated, conventional techniques of pharmaceutical formulating and the like, which are within the skill of the art. Such techniques are fully explained in the literature. See, for example, Remington, The Science and Practice of Pharmacy, supra.

In the following examples, efforts have been made to ensure accuracy with respect to numbers used (e.g., amounts, temperatures, and so forth) but some experimental error and deviation should be accounted for. Unless indicated otherwise, temperature is in degrees C. and pressure is at or near atmospheric pressure at sea level. All reagents were obtained commercially unless otherwise indicated.

The specific objectives of the experimental were to prepare high concentration (>100 mg/mL) formulations of monoclonal antibodies that exhibited one or more of the following properties: processing stability (i.e., >95% monomer remaining following spray drying and reconstitution at a low protein concentration, 5 mg/mL); reconstitution stability (i.e., >95% monomer remaining following spray drying and reconstitution at a high protein concentration, ≧100 mg/mL); time of reconstitution at high concentration of less than 15 minutes; and good syringeability through a narrow (28G) needle, as defined by both ease of syringing, determined empirically, and dosage homogeneity, as defined by no significant change in protein concentration and stability during syringing.

Materials:

Human IgG: Polyclonal Human IgG (Lot# 31K9001) was purchased from Sigma Chemical Company (St. Louis, Mo.). It was supplied as a lyophilized powder essentially free of excipients and was used in this study without any further purification.

CAT-213: CAT-213 is a fully human monoclonal antibody (IgG₄) specific for human eotaxin-1. Eotaxin-1 is a chemokine that specifically attracts eosinophils into tissues where they degranulate, causing tissue damage and inflammation. This occurs in a variety of allergic reactions including asthma and may therefore be useful in treating allergic rhinitis. CAT-213 was received from Cambridge Antibody Technologies (Cambridge, UK). Additional information concerning CAT-213, including a method for preparing CAT-213, is described in WO 01/66754.

Other materials: Citric acid, sodium phosphate monobasic, histidine, trehalose and sucrose were purchased from Sigma Chemical Co. (St. Louis, Mo.). Tween-80 was obtained from JT Baker (Phillipsburg, N.J.) while sodium phosphate dibasic and sodium citrate dihydrate were purchased from Spectrum (Gardena, Calif.) and Mallinckrodt (Paris, Ky.), respectively. All chemicals were of analytical grade (purity ≧95%).

Experimental Methods:

Formulation Preparation: The lyophilized IgG material purchased from Sigma was reconstituted with 1 mM histidine buffer, pH 6.0 to a concentration of 5 mg/mL. Trehalose and Tween-20 were added to the reconstituted protein solution at concentrations of 12 mM and 0.002% w/v, respectively. The resulting mixture was stirred slowly using a magnetic stirrer to obtain a homogeneous solution. The solution was then spray dried according to the method described below.

Diafiltration: The supplied CAT-213 solution was diafiltered to remove salt and replace the existing buffer with a 2 mM sodium citrate buffer, pH 5.6. Diafiltration was performed using a 200 mL Type 8200 Amicon (Amicon Co., Beverly, Mass.) stirred cell apparatus with a YM30K membrane (Millipore Corporation, Bedford, Mass.). The protein solution was stirred constantly using a magnetic stirrer at a low stirring rate to minimize exposure of the protein to shear. The entire operation was carried out under refrigeration (2 to 8° C.) at a pressure of 65 psi. Diafiltration was continued until at least seven times the initial volume of protein solution was recovered in the filtrate, thereby allowing for a seven-fold washing of the starting buffer components. Protein concentration in the retentate was monitored by size exclusion—high performance liquid chromatograph (HPLC), as described below. It should be noted that the final solution appeared slightly cloudy indicating that some of the protein was lost as a precipitate during diafiltration. To quantitatively assess the losses, the precipitate was removed by filtering the final protein solution through a 0.22 μm membrane. The protein concentration and the % monomer remaining in the filtered protein solution were determined by HPLC, as described below. Protein losses during diafiltration ranged between 25 to 30%, and they were reproducible for all diafiltration runs performed.

IgG HPLC Analysis: Size exclusion chromatographic analysis of IgG formulations was performed using a SW_{XL} 4000 (7.8×300 mm) size exclusion column (TosoHaas Biosep, Montgomeryville, Pa.) and a fully automated HP 1050 HPLC system (Hewlett Packard, Palo Alto, Calif.). Typically 15 to 25 μg of protein were loaded onto the column. Elution was performed with a mobile phase consisting of 0.05M potassium phosphate buffer (pH 6.8), 0.1M potassium chloride and 0.0015M sodium nitrite at a flow rate of 1 mL/min. The monomeric protein eluted at approximately 10 minutes and elution was monitored at 280 nm using a Hewlett Packard UV-VIS detector (Palo Alto, Calif.). All samples were run in duplicates and reported results reflect mean values ± one standard deviation. Human IgG concentrations were calculated by extrapolation to a standard curve, which was prepared by reconstituting the lyophilized material with histidine buffer to a 1 mg/mL concentration and injecting it onto the column over a range of 1 to 50 μg (r²≧0.999).

CAT-213 HPLC Analysis: With a few exceptions, HPLC analysis for CAT-213 was performed using the same experimental setup and conditions as described with respect to the IgG HPLC analysis. Instead of a potassium phosphate buffer and monitoring at 280 nm, a 0.2M sodium phosphate pH 7.5 buffer was used as the mobile phase and protein elution was monitored at 220 nm. All samples were run in duplicates and reported results reflect mean values ± one standard deviation. CAT-213 concentrations were calculated by extrapolation to a standard curve, which was prepared by diluting CAT-213 standards with the mobile phase and injecting them onto the column over a range of 1 to 50 μg (r²>0.999).

Spray Drying: The IgG formulation was spray dried using a Büchi spray dryer (Büchi, Switzerland) equipped with a modified nozzle. The formulation was continuously fed to the spray dryer at a flow rate of 5 mg/mL and was dried at an inlet temperature of 70° C. and an atomization pressure of 40 psi. The outlet temperatures ranged from 38-40° C. For CAT-213 formulations, spray drying was achieved by continuously feeding the formulations at a flow rate of 5 mg/mL into the Büchi at an inlet temperature of 115° C. and an atomization pressure of 80 psi. The outlet temperatures under these conditions ranged from 65-67° C.

Processing stability: Following spray drying, powder formulations were reconstituted with the appropriate diluent to a protein concentration similar to that before spray drying (approximately 5 mg/mL). Processing stability of spray-dried IgG and CAT-213 formulations was determined by monitoring the formation of irreversible, soluble aggregates by HPLC, as described above. All IgG formulations were reconstituted in deionized water, while CAT-213 formulations were reconstituted with diluents containing either 0.05% or 0.1% w/v of Tween-80.

Reconstitution Stability: To determine high concentration reconstitution stability, the formulations were reconstituted with the appropriate diluent to target protein concentrations of 50, 70, 100, 150 or 200 mg/mL, as described below. Aliquots of the reconstituted formulations were immediately diluted to a protein concentration of ˜5 mg/mL and analyzed for stability using the HPLC protocols described above. The diluents used for reconstituting IgG and CAT-213 formulations were the same as those used to evaluate processing stability, as described above.

Reconstitution Time Analysis: During the reconstitution process, the time required to reach complete reconstitution, evidenced by achievement of visual clarity, was determined and reported as reconstitution time.

Syringeability Analysis: The high concentration CAT-213 formulations were syringed manually through a 28G needle using a 1 mL tuberculin syringe (Becton Dickinson, Franklin Lakes, N.J.). The ease with which the formulations passed through the needle was assessed manually. Further, formulation homogeneity was determined by recording the protein concentration and % monomer remaining before and after syringing using the HPLC protocol described above.

Insoluble Aggregate Analysis: To determine the presence of large, insoluble aggregates, the UV absorbance of high concentration CAT-213 formulations was monitored at 280, 350 and 400 nm immediately after reconstitution and also following filtration through a 0.22 μm membrane. Potential reduction of the intensity of scattered light before and after filtration, served as an indication of the presence of insoluble aggregates in the unfiltered solution. Further, the light scattering absorbance from untreated and filtered samples was monitored following serial dilutions of the starting samples to protein concentrations of 5, 2.5 and 1.25 mg/mL.

### EXAMPLE 1

### IgG Formulation

A dry powder IgG formulation was prepared. Polyclonal human IgG and trehalose (a glass-forming substance due to it relatively high glass transition temperature) were combined in a trehalose to IgG molar ratio of ˜350:1. Further, a small amount of Tween-20 was added to the formulation to minimize protein aggregation and also to facilitate rapid reconstitution of the spray dried powder. Finally, a histidine buffer was included to enhance stability. The components were spray dried as described above and the resulting spray-dried formulations were tested.

Processing Stability: The human IgG spray-dried formulations were analyzed by HPLC both before and after spray drying (SD), and following reconstitution to low concentration (5 mg/mL). Both the before and after spray-dried formulation were reconstituted with deionized water, % monomer remaining analyzed by HPLC. Each sample was run in duplicate and reported as % mean±standard deviation (n=2).

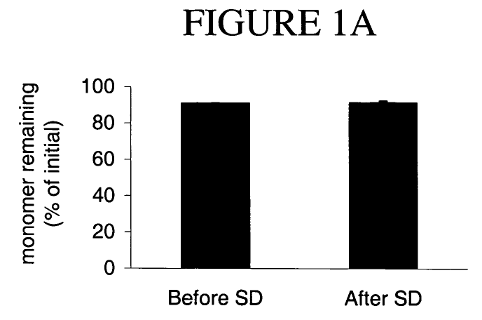

The results are provided in graph form in FIG. 1A, wherein the HPLC analysis indicated the low purity of the starting material (lyophilized human IgG, designated as “Before SD”), consisting of only 91.1±0.7% monomer. Formulating the IgG into a spray-dried powder (designated as “After SD” in FIG. 1A) did not change its purity profile, as indicated by the unaltered fraction of soluble monomer (91.3±1.0%). In addition, the chromatograms provided in FIG. 1B (the lyophilized human IgG formulation) and FIG. 1C (the spray-dried human IgG formulation) show the main impurity for both formulations consisted of a high molecular weight species. This result suggested that human IgG did not undergo any degradation during spray drying and following reconstitution to a low protein concentration, indicating good processing stability.

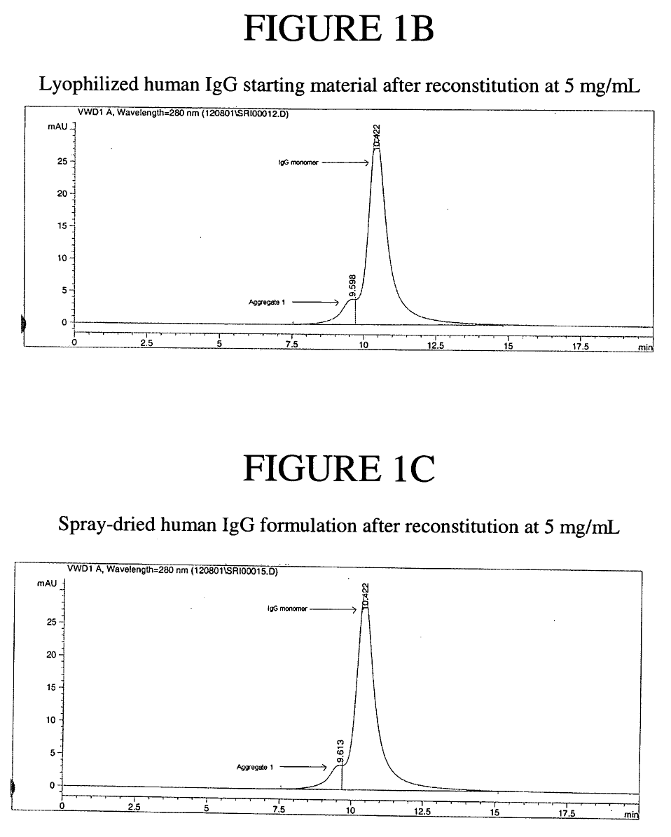

IgG Reconstitution stability: High concentration reconstitution stability determinations for the IgG formulations were conducted and the results are shown in FIG. 2. Again, formulations were reconstituted with deionized water, and the % monomer remaining was analyzed by HPLC. Each sample was run in duplicate and reported as % mean±standard deviation (n=2).

As shown in FIG. 2A, the lyophilized starting material (supplied by Sigma) at a concentration of 5 mg/mL [“Lyophilized (Reconstituted at 5 mg/mL)”] could not maintain its integrity at high concentration [“Lyophilized (Reconstituted at 200 mg/mL”)], as evidenced by the reduction in the amount of soluble monomer to 79.9±0.03% in the formulation designated as “Lyophilized (Reconstituted at 200 mg/mL)”

Moreover, as indicated by the chromatogram provided in FIG. 2B, this reduction in the amount of soluble monomer was due to the formation of higher molecular weight species, eluting around 7.8 minutes. Formation of these higher-order aggregates appears to be concentration-driven, as they were absent at 5 mg/mL. Returning to FIG. 2A, addition of the selected excipients in the spray-dried formulations maintained protein integrity upon reconstitution at 50, 100 and 200 mg/mL. See “Spray Dried” results in FIG. 2A. This was due to the stabilization of the protein monomer, as higher-order aggregates were absent even at a concentration of 200 mg/mL. See the chromatogram provided in FIG. 2C.

The observed difference in stability between the starting material and the spray-dried formulation was due to the protective effects of excipients (trehalose and Tween-20) that were added to the spray-dried formulation. This data show that spray-dried formulations stabilize human IgG after reconstitution to a high protein concentration of 200 mg/mL.

Reconstitution Time Analysis: As indicated by the reconstitution analysis presented in Table II, the spray-dried formulation reconstituted much faster than the corresponding lyophilized starting material at 200 mg/mL. Both formulations were reconstituted with deionized water, as described above.

TABLE II
Reconstitution times for Spray-Dried
and Lyophilized Formulations

| Formulation | Reconstitution Time (minutes) |
|---|---|
| Spray dried | <5 |
| Lyophilized | 11 |

### EXAMPLES 2-8

### CAT-213 Formulations

Three CAT-213 formulations were prepared. The composition of each formulation is provided in Table III. Formulations were reconstituted at low concentrations (5 mg/mL) with diluents containing varying amounts of Tween-80: Diluent A containing 0.05% and Diluent B containing 0.1% w/v.

TABLE III
Composition Description of Preliminary CAT-213 Formulations

| | Carbohydrate | CAT-213 | Carbohydrate | Citrate Buffer | Tween-80 |
|---|---|---|---|---|---|
| Formulation # | Type | (% w/w) | (% w/w) | (% w/w) | (% w/w) |
| 1 | Trehalose | 49 | 45 | 6 | 0 |
| 2 | Sucrose | 51 | 43 | 6 | 0 |
| 3 | Trehalose | 49 | 45 | 5.3 | 0.7 |

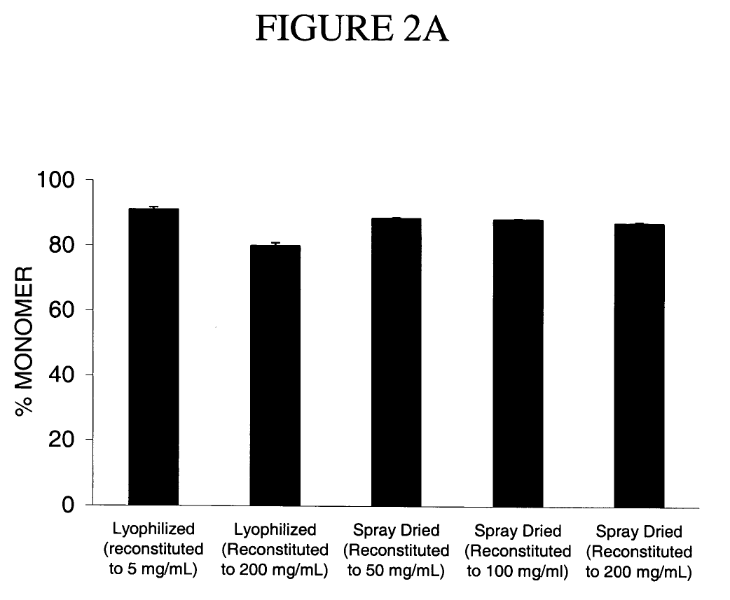

The results of the processing stability analysis are shown, as % monomer remaining following spray drying, in FIG. 3. Sample analysis was preformed in duplicates and results represent means ± one standard deviation. All three CAT-213 formulations retained their stability following spray drying and reconstitution at low concentration. These results are in agreement with those obtained with human IgG, indicating that spray-drying technology presents a very robust formulation approach.

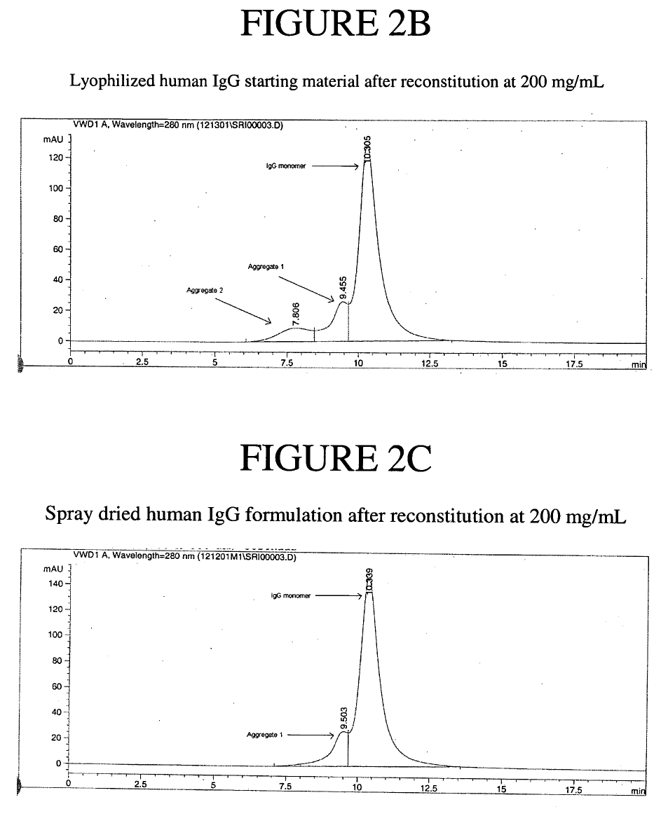

To further assess their integrity, all three CAT-213-containing formulations were evaluated using SEC-HPLC. Sample analyses were performed in duplicate and the results represent means ± one standard deviation. A 5 mg/mL formulation prior to spray drying was analyzed, followed by the three spray dried formulations that were each reconstituted to 70 mg CAT-213/mL with Diluent A, 70 mg CAT-213/mL with Diluent B; and 150 mg/mL with Diluent B. The results are shown in FIG. 4.

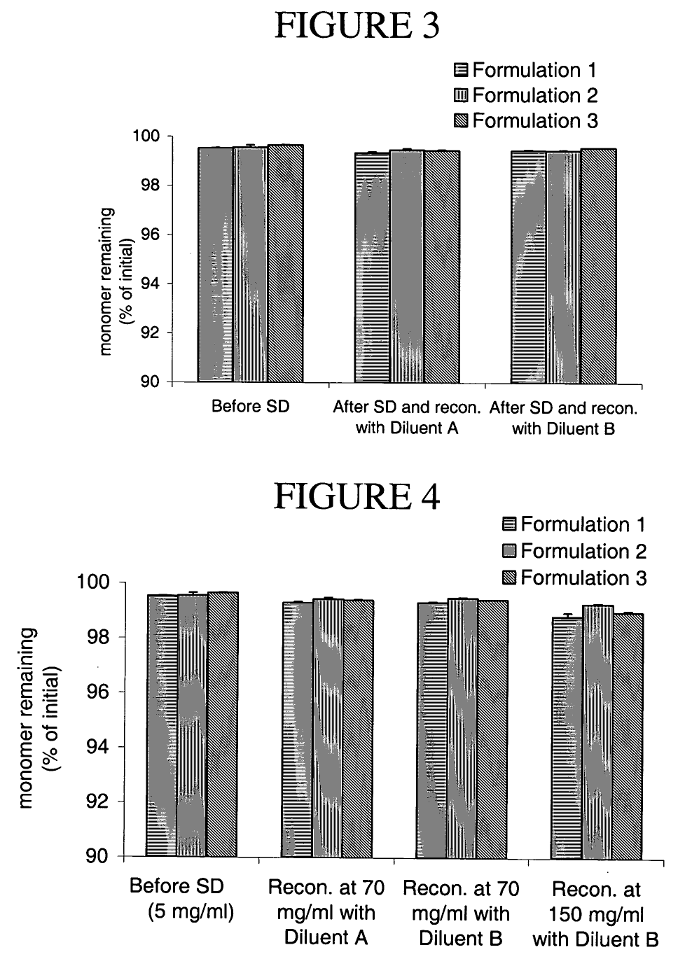

Turning to FIG. 4, the SEC-HPLC data indicate that CAT-213 retained its highly monomeric status in the spray-dried formulations upon reconstitution at 70 mg/mL (99.3 to 99.5%) in all three formulations, regardless of the type of diluent used. Even at a concentration of 150 mg/mL, all three formulations exhibited good stability, as indicated by the virtually unchanged fraction monomer remaining (98.8 to 99.3%) following spray drying and reconstitution at high temperature. The chromatograms corresponding to a stock CAT-213 formulation and a reconstituted CAT-213 formulation at 190 mg/mL are provided in FIGS. 5A and 5B, respectively.

Having demonstrated ease of processing and reconstitution stability, formulations were also evaluated for reconstitution time at high concentration.

Reconstitution time was assessed for formulations 2 and 3 at a CAT-213 concentration of 100 mg/mL with Diluent B and the results are given in Table IV. Formulation 1 was not analyzed due to lack of sufficient sample size. Formulations were reconstituted with Diluent B and analyzed by HPLC. Sample analyses were performed in duplicates and results represent means ± one standard deviation.

TABLE IV
Reconstitution Times for Formulations 2 and 3

| Formulation # | Reconstitution time (minutes) |
|---|---|
| 2 | >10 |
| 3 | <4 |

Both formulations reconstituted to form clear, non-viscous solutions, suggesting the lack of presence of any large insoluble aggregates even at a high protein concentration. Both formulations were reconstituted in less than 15 minutes; formulation 2 reconstituted in over 10 minutes; while formulation 3 reconstituted in a short time (<4 minutes). While not wishing to be bound by theory, it may be that the surfactant included in formulation 3 (added prior to spray drying) facilitated faster reconstitution as compared to adding the surfactant only during the reconstitution stage. In addition, the surfactant within the powder may greatly facilitate its wetting with a diluent that also contained a surfactant. These results are in agreement with literature studies, which also demonstrated the utility of surfactants, in enabling fast reconstitution of lyophilized powders. See, for example, Webb et al. (2001) Anal. Chem. 73(21):5296-301.

The syringeability of formulations 2 and 3 was further evaluated at a concentration of 100 mg/mL, per the method given in above. Formulation 1 was not analyzed due to lack of sufficient sample size. The results are given in Table V. Both formulations passed effortlessly through a 28G needle. No significant change in CAT-213 concentration and % monomer remaining before and after syringing was observed, suggesting that the formulations maintained their homogeneity and stability during syringing, thereby meeting the target for ease and homogeneity of syringeability.

TABLE V
Syringeability Analysis for Formulations 2 and 3

| | CAT-213 (mg/mL) | | % Monomer Remaining | |
|---|---|---|---|---|
| | Start of | End of | Start of | End of |
| Formulation # | syringing | syringing | syringing | syringing |
| 2 | 98.5 ± 0.2 | 98.3 ± 0.7 | 99.0 ± 0.1 | 98.8 ± 0.2 |
| 3 | 96.5 ± 1.2 | 97.5 | 97.1 ± 0.0 | 98.1 |

High Concentration Formulation Reproducibility To establish reproducibility, additional CAT-213 formulations were formulated. Formulations comprising internal surfactant with the disaccharide sucrose (Formulation #4) or trehalose (Formulation #5). The exact compositions are given in Table VI.

TABLE VI
Composition Description of High Concentration CAT-213 Formulations

| Formulation | Carbohydrate | CAT-213 | Carbohydrate | Citrate Buffer | Tween-80 |
|---|---|---|---|---|---|
| # | Type | (% w/w) | (% w/w) | (% w/w) | (% w/w) |
| 4 | Trehalose | 53 | 41 | 5.4 | 0.6 |
| 5 | Sucrose | 55 | 39 | 5.4 | 0.6 |

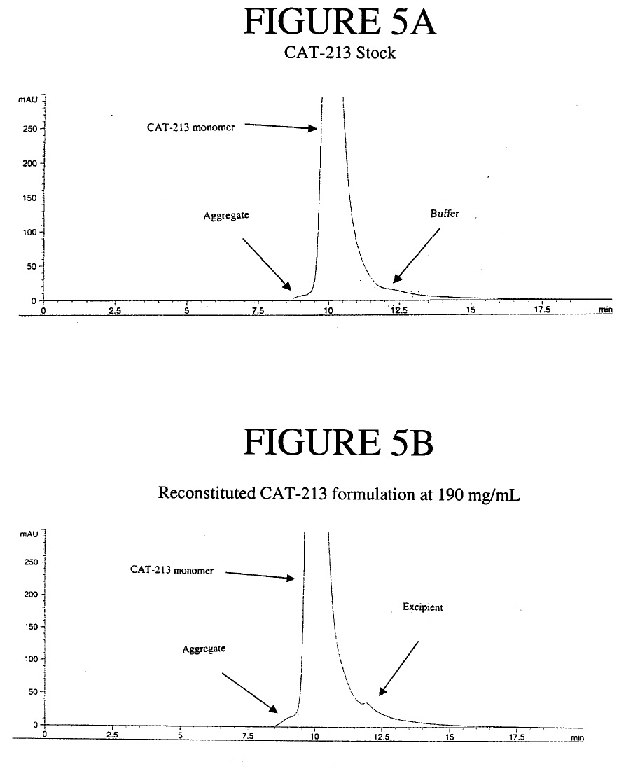

SEC-HPLC analysis of the CAT-213 formulations before & after spray drying (SD), and following reconstitution at 140 mg/mL with 0.1% w/v Tween-80 were conducted. The results of the reconstitution analysis at 140 mg/mL, shown in FIG. 6, indicated that there was no change in % monomer remaining in both formulations. These results confirmed the earlier data, thereby demonstrating the performance reproducibility of the spray-dried formulations. Further, both formulations reconstituted within 5 minutes (Table VII) to form clear non-viscous solutions, supporting the conclusion that the formulations lacked any large insoluble aggregates. The formulations were reconstituted with Diluent B; formulation 5 was not analyzed at 190 mg/mL due to lack of sample size.

TABLE VII
Reconstitution times for high concentration CAT-213 formulations

| Formulation | Reconstitution time | Reconstitution time |
|---|---|---|
| # | 140 mg/mL (minutes) | 190 mg/mL (minutes) |
| 4 | <5 | >10 |
| 5 | <5 | — |

Having established performance reproducibility, formulation performance at 190 mg/mL, was assessed. CAT-213-containing formulation 4 was analyzed before and after spray drying (SD), and after spray drying and reconstitution at 190 mg/mL with 0.1% w/v Tween-80. formulation 5 could not be analyzed due to lack of sample size. The sample analyses were performed in duplicate and the results represent means ± one standard deviation.

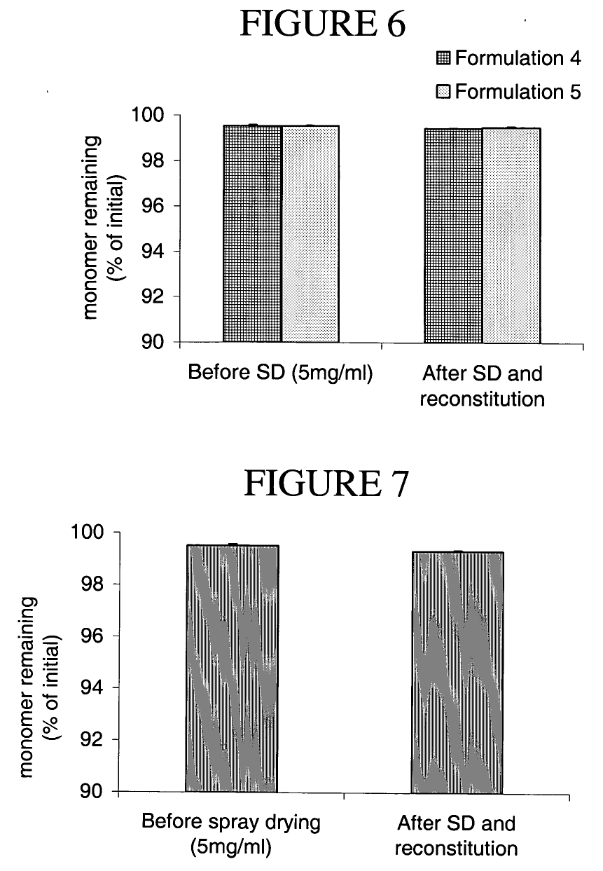

The results of % monomer change analysis are shown in FIG. 7, while the reconstitution time is given in Table VII (above). The data indicate that there was no significant change in % monomer remaining in the formulation following processing and reconstitution as compared to that before spray drying, suggesting that, similarly with IgG, the stability of CAT-213 was maintained even at 190 mg/mL, albeit exhibiting a slower reconstitution (approximately 10 minutes). Even with though the formulation's increased viscosity increased the reconstitution time, the formulation nonetheless was deemed to be pharmaceutically acceptable.

The presence of minor insoluble aggregates in formulation 4 at 190 mg/mL was further investigated using the UV turbidity assay described above (see insoluble aggregate analysis). Absorbances for the filtered and unfiltered samples are shown in Table VIII.

TABLE VIII
Insoluble Aggregate Analysis of High
Concentration CAT-213 Formulation 4

| Concentration | Unfiltered sample | | | Filtered sample | | |
|---|---|---|---|---|---|---|
| (mg/mL) | A_{400 nm} | A_{350 nm} | A_{280 nm} | A_{400 nm} | A_{350 nm} | A_{280 nm} |
| 5.0 | 0.05 | 0.08 | 3.85 | 0.02 | 0.04 | 3.81 |
| 2.5 | 0.03 | 0.04 | 3.59 | 0.01 | 0.02 | 3.55 |
| 1.25 | 0.02 | 0.03 | 1.94 | 0.02 | 0.02 | 1.92 |

The lack of any significant difference in the scattering intensity at 350 and 400 nm between filtered and unfiltered samples (at the same concentration), indicated the absence of light scattering resulting from the presence of large insoluble particles. This may provide corroborative evidence for the absence of insoluble aggregates in the reconstituted formulation at a high concentration. However, it does not rule out the possibility that smaller insoluble particles may be present.

Viscosity Reducing Formulations Although all formulations were deemed acceptable, an attempt was made to enable the faster reconstitution of formulations 4 and 5 at high concentrations. It was hypothesized that reducing viscosity might provide a faster reconstitution time as it appeared that higher viscosities resulted in somewhat longer reconstitution times. Since the viscosity-enhancing effect of sugars at high concentrations is well documented (see, for example, Mazurkiewicz et al. (1998) Pol. J. Food Nutr. Sci. 7(48):171-180), formulations were designed with decreasing amounts of sucrose. The compositions of two viscosity-reducing formulations, formulations 6 and 7, are given in Table IX.

TABLE IX
Composition Description of Viscosity Reducing CAT-213 Formulations

| | Carbohydrate | CAT-213 | Carbohydrate | Citrate Buffer | Tween-80 |
|---|---|---|---|---|---|
| Formulation # | Type | (% w/w) | (% w/w) | (% w/w) | (% w/w) |
| 6 | Sucrose | 78 | 13 | 8 | 1 |
| 7 | Sucrose | 67 | 25 | 7 | 1 |

Both formulations reconstituted quickly (<10 minutes) at 190 mg/mL (using Diluent B) to form clear solutions, thereby confirming that the high viscosity and slower reconstitution time was due to the presence of relatively excess sugar. The reconstitution times for the two formulations are given in Table X.

TABLE X
Reconstitution Times for Viscosity Reducing
CAT-213 for Formulations 6 and 7

| Formulation # | Reconstitution time (minutes) |
|---|---|
| 6 | <10 |
| 7 | <10 |

The CAT-213-containing formulations were analyzed before and after spray drying (SD), and following reconstitution at 190 mg/mL with 0.1% w/v Tween-80 by SEC-HPLC for % monomer. Sample analyses were performed in duplicate and the results represent means +one standard deviation.

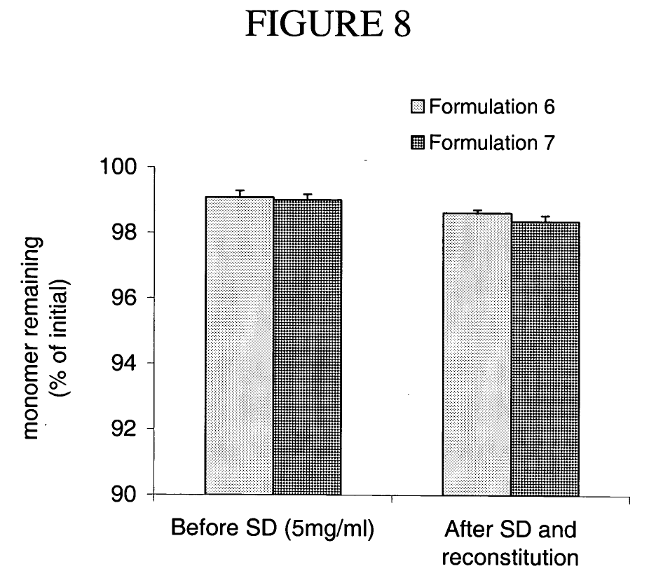

The results, shown in FIG. 8, indicated no change in % monomer remaining following spray drying and reconstitution as compared to that before spray drying, suggesting that decreasing the carbohydrate content in the formulation by 2- and 4-fold respectively, does not have a major impact on protein stability.

Conclusions: These results demonstrate that antibodies can be formulated successfully as spray-dried, dry powders. The dry powder formulations can be reconstituted quickly to high protein concentrations without loss of protein stability and the reconstituted formulations can be syringed easily through a narrow gauge needle while maintaining their homogeneity during syringing. In summary, the experimental demonstrated that spray-drying technology can produce antibody formulations that meet all performance standards required for subcutaneous administration, thereby enabling delivery of these molecules via this route.

1. A composition comprising antibody-containing particles, wherein the particles have a mass median diameter of greater than 7.5 μm and less than 100 μm.

2. The composition of claim 1, wherein the particles have a mass median diameter of greater than 10 μm and less than 100 μm.

3. The composition of claim 1, wherein the antibody is an antibody fragment.

4. The composition of claim 3, wherein the antibody fragment is selected from the group consisting of Fab, F(ab)₂, Fv, and single polypeptide chain binding molecule.

5. The composition of claim 1, wherein the antibody is a full-length antibody.

6. The composition of claim 1, wherein the antibody is murine.

7. The composition of claim 1, wherein the antibody is chimeric.

8. The composition of claim 1, wherein the antibody is CDR-grafted.

9. The composition of claim 1, wherein the antibody is humanized.

10. The composition of claim 1, wherein the antibody is an antibody-conjugate.

11. The composition of claim 1, wherein the antibody or antibody fragment is a type selected from the group consisting of IgE, IgG, and IgM.

12. The composition of claim 11, wherein the antibody is an IgG-type.

13. The composition of claim 1, further comprising a pharmaceutically acceptable excipient.

14. The composition of claim 13, wherein the pharmaceutically acceptable excipient is present in the antibody-containing particles.

15. The composition of claim 13, wherein the pharmaceutically acceptable excipient is comprised of particles separate and distinct from the antibody-containing particles.

16. The composition of claim 13, wherein the excipient is selected from the group consisting of amino acid, amino acid derivative, oligopeptide, carbohydrate, inorganic salts, antimicrobial agents, antioxidants, surfactants, buffers, acids, bases, and combinations thereof.

17. The composition of claim 16, wherein the excipient is a carbohydrate.

18. The composition of claim 17, wherein the carbohydrate is selected from the group consisting of fructose, maltose, galactose, glucose, mannose, sorbose, lactose, sucrose, trehalose, cellobiose, raffinose, melezitose, maltodextrans, dextrans, starches, mannitol, xylitol, lactitol, glucitol, pyranosyl sorbitol, myoinositol, and combinations thereof.

19. The composition of claim 17, wherein the carbohydrate is selected from the group consisting of sucrose and trehalose.

20. The composition of claim 16, wherein the excipient is selected from a salt or buffer.

21. The composition of claim 20, wherein the salt or buffer is selected from the group consisting of citric acid, sodium phosphate monobasic, sodium phosphate dibasic, and combinations thereof.

22. The composition of claim 16, wherein the excipient is a surfactant.

23. The composition of claim 22, wherein the surfactant is selected from the group consisting of Tween-20, Tween-80, and combinations thereof.

24. The composition of claim 16, wherein the excipient is an amino acid.

25. The composition of claim 24, wherein the amino acid is selected from the group consisting of leucine, histidine, and combinations thereof.

26. The composition of claim 1, wherein the composition is housed in a syringe.

27. The composition of claim 1, wherein the composition is housed in a vial.

28. The composition of claim 1, wherein the antibody is noncrystalline.

29. The composition of claim 1, wherein the antibody is partially amorphous.

30. The composition of claim 1, having substantially no aggregates.

31. A reconstituted composition comprising an antibody in an amount of from about 25 mg/mL to about 1000 mg/mL, a diluent and an optional excipient, wherein the reconstituted composition is formed from a spray-dried powder comprised of the antibody or antibody fragment and the optional excipient.

32. The composition of claim 31, in sterile form.

33. The composition of claim 31, wherein the antibody is an antibody fragment.

34. The composition of claim 33, wherein the antibody fragment is selected from the group consisting of Fab, F(ab)₂, Fv, and single polypeptide chain binding molecule.

35. The composition of claim 31, wherein the antibody is a full-length antibody.

36. The composition of claim 31 wherein the antibody is murine.

37. The composition of claim 31 wherein the antibody is chimeric.

38. The composition of claim 31 wherein the antibody is CDR-grafted.

39. The composition of claim 31 wherein the antibody is humanized.

40. The composition of claim 31 wherein the antibody is an antibody-conjugate.

41. The composition of claim 31 wherein the antibody or antibody fragment is a type selected from the group consisting of IgE, IgG, and IgM.

42. The composition of claim 41, wherein the antibody is an IgG-type.

43. The composition of claim 31, wherein the pharmaceutically acceptable excipient is present.

44. The composition of claim 43, wherein the excipient is selected from the group consisting of amino acid, amino acid derivative, oligopeptide, carbohydrate, inorganic salts, antimicrobial agents, antioxidants, surfactants, buffers, acids, bases, and combinations thereof.

45. The composition of claim 44, wherein the excipient is a carbohydrate.

46. The composition of claim 45, wherein the carbohydrate is selected from the group consisting of fructose, maltose, galactose, glucose, mannose, sorbose, lactose, sucrose, trehalose, cellobiose, raffinose, melezitose, maltodextrans, dextrans, starches, mannitol, xylitol, lactitol, glucitol, pyranosyl sorbitol, myoinositol, and combinations thereof.

47. The composition of claim 45, wherein the carbohydrate is selected from the group consisting of sucrose and trehalose.

48. The composition of claim 44, wherein the excipient is selected from a salt or buffer.

49. The composition of claim 48, wherein the salt or buffer is selected from the group consisting of citric acid, sodium phosphate monobasic, sodium phosphate dibasic, and combinations thereof.

50. The composition of claim 44, wherein the excipient is a surfactant.

51. The composition of claim 50, wherein the surfactant is selected from the group consisting of Tween-20, Tween-80, and combinations thereof.

52. The composition of claim 44, wherein the excipient is an amino acid.

53. The composition of claim 52, wherein the amino acid is selected from the group consisting of leucine, histidine, and combinations thereof.

54. The composition of claim 31, wherein the composition is housed in a syringe.

55. The composition of claim 31, wherein the composition is housed in a vial.

56. The composition of claim 31, wherein the diluent is selected from the group consisting of bacteriostatic water for injection, dextrose 5% in water, phosphate-buffered saline, Ringer's solution, saline, sterile water, deionized water, and combinations thereof.

57. The composition of claim 31, wherein the optional excipient is present.

58. The composition of claim 31, wherein the antibody is present in an amount of from about 25 mg/mL to about 250 mg/mL.

59. The composition of claim 31, having substantially no aggregates.

60. A method for preparing a reconstituted composition comprising the steps of providing a spray-dried powder comprised of an antibody and adding a diluent in order to form the reconstituted composition, wherein the antibody is present in the reconstituted composition in an amount of from about 25 mg/mL to about 1000 mg/mL.

61. The method of claim 60, wherein the reconstituted composition comprises an excipient.

62. The method of claim 61, wherein the excipient is present in the spray-dried powder.

63. The method of claim 61, wherein the excipient is added with or after the step of adding the diluent.

64. The method of claim 60, wherein the step of providing the spray-dried powder is comprised of combining the antibody in a liquid to form a liquid feed and spray drying the liquid feed to form the spray-dried powder.

65. The method of claim 60, wherein the reconstituted composition has substantially no aggregates.

66. The method of claim 60, wherein the reconstituted composition becomes visually clear within about 15 minutes of adding the diluent.

67. The method of claim 66, wherein the reconstituted composition becomes visually clear within about 10 minutes of adding the diluent.

68. The method of claim 67, wherein the reconstituted composition becomes visually clear within about 5 minutes of adding the diluent.

69. The method of claim 60, wherein the diluent is selected from the group consisting of diluent is selected from the group consisting of bacteriostatic water for injection, dextrose 5% in water, phosphate-buffered saline, Ringer's solution, saline, sterile water, deionized water, and combinations thereof.

70. The method of claim 60, wherein the antibody is present in the reconstituted composition in an amount of from about 25 mg/mL to about 250 mg/mL.

71. A method of administering a composition to a patient comprising administering, via injection, a therapeutically effective amount of an antibody present in a reconstituted composition, wherein the reconstituted composition is comprised of an antibody concentration of from about 25 mg/mL to about 1000 mg/mL, a diluent and an optional excipient, wherein the reconstituted composition is formed from a spray-dried powder comprised of the antibody and the optional excipient.

72. The method of claim 71, wherein the injection is a subcutaneous injection.

73. The method of claim 71, wherein the injection is an intramuscular injection.

74. The method of claim 71, wherein the injection is an intravenous injection.
